(19) United States
(12) Plant Patent Application Publication
(10) Pub. No.: US 2006/0230477 P1
(43) Pub. Date: Oct. 12, 2006

(54) Variety of rose plant named 'Dream White'

(50) ROSA HYBRIDA; CLIMBING ROSE

(76) Inventor: Clive Wallis, Dunedin (NZ)

Correspondence Address: FULWIDER PATTON LEE & UTECHT, LLP, 200 OCEANGATE, SUITE 1550, LONG BEACH, CA, 90802, US

(21) Appl. No.: 11/101,346

(22) Filed: Apr. 7, 2005

(51) Int. Cl. A01H 5/00

(52) U.S. Cl. PLT/110

(57) ABSTRACT

Plants of the new Rose variety can be compared to other plants of its genus. The habit of ‘Dream White’ is compact and bushy as opposed to open and spreading, and it possesses a larger flower size and light fragrance along with an extended flowering season. It further possesses a vigorous growth habit and can be trained as a climber. The foregoing traits have been repeatedly observed and are determined to be among the unique characteristics of ‘Dream White’.

### BACKGROUND OF THE NEW PLANT

The present invention relates to a new and distinct cultivar of Rose, botanically known as Rosa and further known by the varietal name ‘Dream White.’ This new variety is also sold under the Breeder's reference name ICEBERG SUPREME. The new variety was discovered through spontaneous mutation from Rose Climbing Iceberg. The parental cultivar is sold under the Breeder's reference name ‘Climbing Iceberg.’ The variety was isolated by the inventor, Clive Wallis, in Mosgiel, New Zealand. Cuttings were taken from the selected plant and were grown on to assure stability. Plants were grown over many generations and all have been stable. Subsequent propagation by cuttings in New Zealand and Australia has also proved stable, and the desirable features of the plant have remained constant throughout the evaluation period.

### SUMMARY OF THE INVENTION

Plants of the new Rose variety can be compared to other plants of its genus. The habit of ‘Dream White’ is compact and bushy as opposed to open and spreading and it possesses a larger flower size along with an extended flowering season. It further possesses a vigorous growth habit and can be trained as a climber. The foregoing traits have been repeatedly observed and are determined to be among the unique characteristics of ‘Dream White’. These characteristics distinguish ‘Dream White’ as a new and distinct cultivar:

### DESCRIPTION OF THE DRAWING

The accompanying colored photographs illustrate the new cultivar:

FIG. 1 shows an enlarged view of the flower;

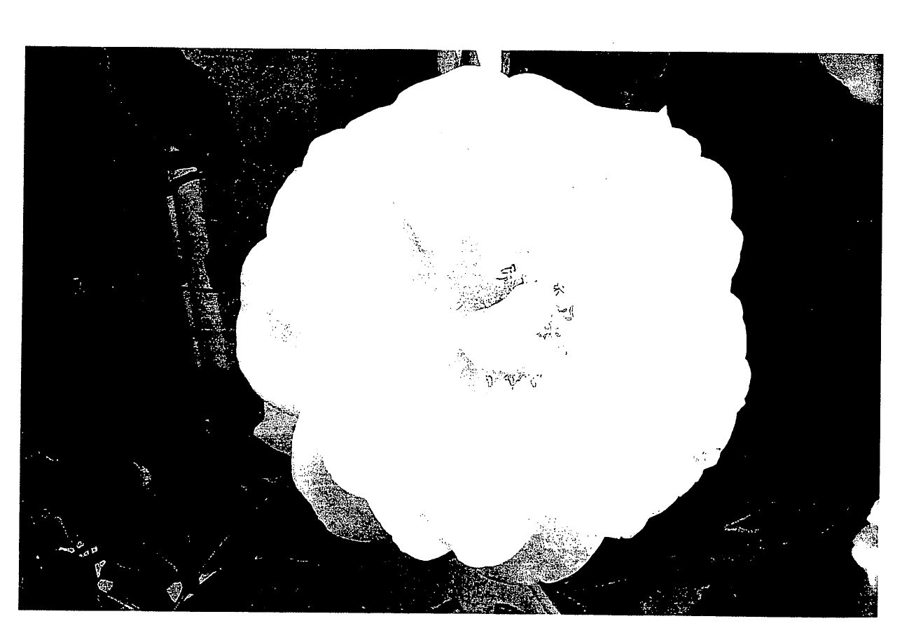

FIG. 2 shows a second enlarged view of the flower;

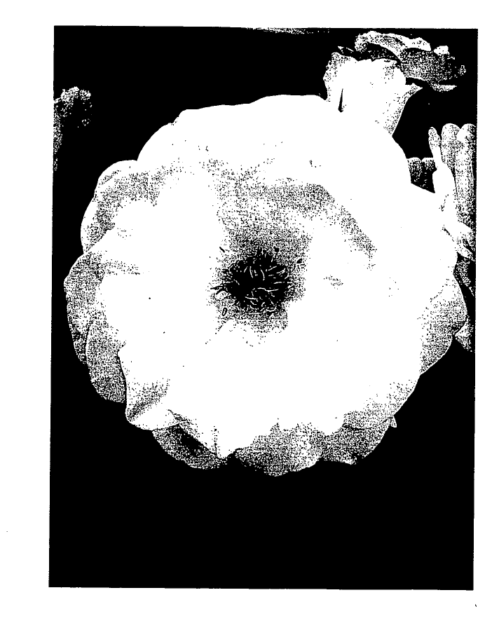

FIG. 3 shows a portion of the variety showing a grouping of the flowers; and

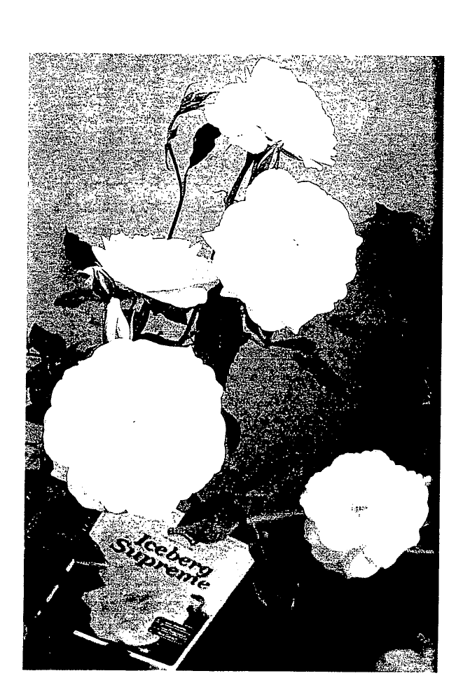

FIG. 4 shows the full variety in a potted reservoir.

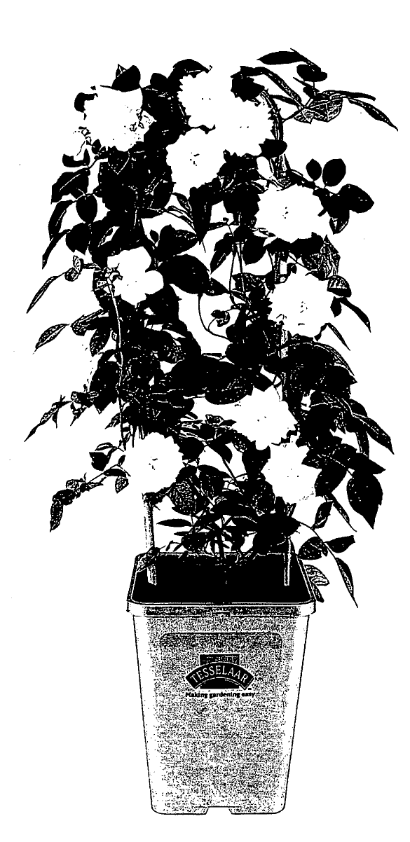

### DETAILED BOTANICAL DESCRIPTION OF THE NEW PLANT

The following detailed description sets forth characteristics of the new cultivar. The data which defines each characteristic was collected from nurseryman Clive Wallis, and carried out by the breeder at Mosgiel, New Zealand.

In the following table of comparative features, color references are made to The Royal Horticultural Society of London Colour Chart, 1995 Edition. The color values were determined on plant material grown in an open field at Mosgiel, New Zealand.
Botanical name: Rosa
Varietal name: ‘Dream White’
Parentage: Rosa hybrida ‘Climbing Iceberg’
The plant:
   Density.—bushy.
   Growth type.—Tall shrub, climbing.
   Growth rate.—extremely vigorous.
   Height.—4 feet.
   Width.—3 feet.
Leaf:
   Color-main.—dark green.
Inflorescence:
   Length of flowering period.—Spring to Early Winter.
   Flower size.—Medium.
   Flower color.—White.
   Fragrance of bloom.—Light.
   Inflorescence type.—Double.
Flower bud:
   Color.—Deep Blush.

### GENERAL CHARACTERISTICS

abundance of perfumed flowers that start out as pink-tinged buds, opening to a pure white flower. It flowers continuously from spring to early winter, and its attributes include long flowering, more flowers, and improved fragrance compared to the parent Rosa ‘Climbing Iceberg.’

What is claimed is:

1. A new and distinct variety of Rose plant, substantially as shown and described.
